(19) United States
(12) Patent Application Publication
(10) Pub. No.: US 2023/0025274 A1
(43) Pub. Date: Jan. 26, 2023

(54) SLEEPING MOUTHPIECE

(71) Applicant: Won Young PARK, Seoul (KR)

(72) Inventor: Won Young PARK, Seoul (KR)

(21) Appl. No.: 17/381,177

(22) Filed: Jul. 20, 2021

(51) Int. Cl. A61F 5/56

(52) U.S. Cl. CPC A61F 5/566

(57) ABSTRACT

Disclosed herein is a sleeping mouthpiece which can prevent separation during sleeping by wrapping the palate such that the sleeping mouthpiece gets in contact with the palate and can be adjusted according to the teeth arrangement by adjusting an upper mouth fit to the upper teeth and a lower mouth fit to the lower teeth back and forth. The sleeping mouthpiece includes: an upper mouth having an upper outer guide, an upper inner guide and an upper occlusal surface to form an upper teeth fitting part to which the upper teeth are fit, so that an upper rigid panel is inserted into the upper mouth; and a lower mouth having a lower outer guide, a lower inner guide and a lower occlusal surface to form a lower teeth fitting part to which the lower teeth are fit, so that a lower rigid panel is inserted into the lower mouth, wherein a hard palate support is connected to the upper end of the upper inner guide and comes into contact with the intraoral hard palate to support the intraoral hard palate, sliding rods are formed at both sides of the upper occlusal surface in order to connect the upper mouth and the lower mouth with each other so that the upper mouth and the lower mouth can be moved back and forth, and the sliding rods are inserted and guided into guide slits formed at both sides of the lower occlusal surface. The sleep mouthpiece can prevent separation of the upper mouth by the power of the tongue to push out of awareness during sleeping since supporting the intraoral hard palate while getting in contact with the intraoral hard palate, and can be adjusted according to various kinds of teeth arrangements since the upper mouth and the lower mouth can move back and forth by coupling of the sliding rods and the guide slits.

### TECHNICAL FIELD

The present invention relates to a sleeping mouthpiece, and more particularly, to a sleeping mouthpiece which can prevent separation during sleeping by wrapping the palate such that the sleeping mouthpiece gets in contact with the palate and can be adjusted according to the teeth arrangement by adjusting an upper mouth fit to the upper teeth and a lower mouth fit to the lower teeth back and forth.

### BACKGROUND

As is generally known, snoring is a breathing noise generated when a respiratory flow in a user's sleep sets up a vibration to the soft palate (a relative soft back part of the palate) which is relaxed while passing the airway narrowed by various causes and to structures around the uvulas which droop down at the center of the soft palate of the mouth.

Moreover, sleep apnea is a frequently respiratory arrest during sleep, and may cause severe snoring, excessive daytime sleepiness, or the likes. Hypoxia generated during sleeping due to the respiratory arrest may cause various complications related with the cardiopulmonary vascular system.

Methods to prevent the snoring and the sleep apnea are divided into surgery operation, a method using a positive pressure supply device and a method using an oral device.

Recently, the method using the oral device which is more simple and more inexpensive than the surgical operation, which requires lots of time for anesthesia and surgery, and the method using the positive pressure supply device which is expensive and is uncomfortable to wear an oxygen mask.

FIGS. 7 and 8 illustrate a conventional oral device. Such a conventional art includes maxillary insertion parts 1 and 10 to which the upper teeth are inserted and mandibular insertion parts 1 and 20 to which the lower teeth are inserted, and the maxillary insertion parts and the mandibular insertion parts are united with each other. Therefore, the conventional art has a disadvantage in that it is deteriorated in effect to prevent snoring and sleep apnea.

Furthermore, because the conventional oral device is fit up to the teeth and the gum, the oral device fit to the teeth may be twisted by power to push out of the teeth with the tongue out of awareness during sleeping or may be separated from the teeth. Therefore, the conventional art has a disadvantage in that it is deteriorated in effect to prevent snoring and sleep apnea.

### SUMMARY OF THE INVENTION

Accordingly, the present invention has been made to solve the above-mentioned problems occurring in the prior arts, and it is an object of the present invention to provide a sleeping mouthpiece which can prevent separation during sleeping by wrapping the palate such that the sleeping mouthpiece gets in contact with the palate and can be adjusted according to the teeth arrangement by adjusting an upper mouth fit to the upper teeth and a lower mouth fit to the lower teeth back and forth.

To accomplish the above object, according to the present invention, there is provided a sleeping mouthpiece including: an upper mouth having an upper outer guide, an upper inner guide and an upper occlusal surface to form an upper teeth fitting part to which the upper teeth are fit, so that an upper rigid panel is inserted into the upper mouth; and a lower mouth having a lower outer guide, a lower inner guide and a lower occlusal surface to form a lower teeth fitting part to which the lower teeth are fit, so that a lower rigid panel is inserted into the lower mouth, wherein a hard palate support is connected to the upper end of the upper inner guide and comes into contact with the intraoral hard palate to support the intraoral hard palate, sliding rods are formed at both sides of the upper occlusal surface in order to connect the upper mouth and the lower mouth with each other so that the upper mouth and the lower mouth can be moved back and forth, and the sliding rods are inserted and guided into guide slits formed at both sides of the lower occlusal surface.

Moreover, the front sides of the guide slits are formed with a slope of 2° to 4° toward the center of the upper mouth.

Furthermore, the guide slits are formed in zigzags in a longitudinal direction.

Additionally, an outer end portion of the hard palate support is smoothly curved toward both ends from the center of the upper end of the upper inner guide, and an inner face of the hard palate has a curvature continued from the inner face of the upper inner guide to the hard palate.

In addition, a plurality of upper nonslip ribs which are elongated vertically are formed at the lower portions of both sides of the exterior of the upper outer guide to get in contact with both sides of the intraoral cheek.

Moreover, a plurality of lower nonslip ribs which are elongated vertically are formed at the upper portions of both sides of the exterior of the lower outer guide to get in contact with both sides of the intraoral cheek.

The sleeping mouthpiece according to the present invention can prevent separation of the upper mouth by the pushing power of the tongue out of awareness during sleeping since the hard palate support comes into contact with the intraoral hard palate to support the intraoral hard palate, and can be adjusted according to various forms of the teeth arrangement since the upper mouth and the lower mouth move back and forth by a combination of the guide slit and the sliding rod.

Additionally, the sleeping mouthpiece according to the present invention is not only effective in preventing snoring and sleep apnea but also effective in preventing teeth grinding and open mouth.

### BRIEF DESCRIPTION OF THE DRAWINGS

The above and other objects, features and advantages of the present invention will be apparent from the following detailed description of the preferred embodiments of the invention in conjunction with the accompanying drawings, in which:

FIG. 1 is a perspective view showing a combined state of an upper mouth and a lower mouth of a sleep mouthpiece according to a preferred embodiment of the present invention;

FIG. 2 is an exploded perspective view showing an exploded state of the upper mouth and the lower mouth of the sleep mouthpiece according to the preferred embodiment of the present invention;

FIG. 3 is a side view of the sleep mouthpiece according to the preferred embodiment of the present invention;

FIG. 4 is a plan view of the sleep mouthpiece according to the preferred embodiment of the present invention;

FIG. 5 is a sectional view taken along the line of A-A of FIG. 4;

FIG. 6 is a bottom side view of the sleep mouthpiece according to the preferred embodiment of the present invention;

FIGS. 7 and 8 are exemplary views of a conventional art.

### DETAILED DESCRIPTION OF THE PREFERRED EMBODIMENT

In order to fully understand the present invention, exemplary embodiments of the invention will be described with reference to the accompanying drawings.

The embodiments of the present invention may be modified in many different forms and the scope of the invention should not be limited to the embodiments set forth herein. Rather, these embodiments are provided so that this disclosure will be thorough and complete, and will fully convey the concept of the invention to those skilled in the art.

In the drawings, the shapes and dimensions may be exaggerated for clarity, and the same reference numerals will be used throughout to designate the same or like components. A detailed explanation of known related functions and constitutions may be omitted to avoid unnecessarily obscuring the subject matter of the present invention.

Hereinafter, a preferred embodiment of the present invention will be described in detail with reference to the accompanying drawings.

The sleep mouthpiece according to the preferred embodiment of the present invention includes: an upper mouth 100 having an upper outer guide 120, an upper inner guide 130 and an upper occlusal surface 140 to form an upper teeth fitting part 110 to which the upper teeth are fit; and a lower mouth 200 having a lower outer guide 220, a lower inner guide 230 and a lower occlusal surface 240 to form a lower teeth fitting part 210 to which the lower teeth are fit.

The upper mouth 100 has the upper teeth fitting part 110 to which the upper teeth including the front teeth, the canine teeth and the back teeth of the upper gum are fit. Such an upper mouth 100 is improved in hygiene and does not make wound in the mouth since being made of ethylene vinyl acetate (EVA).

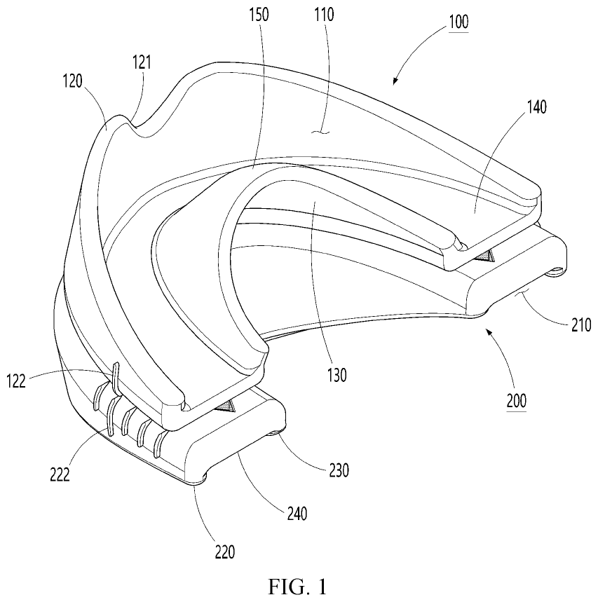

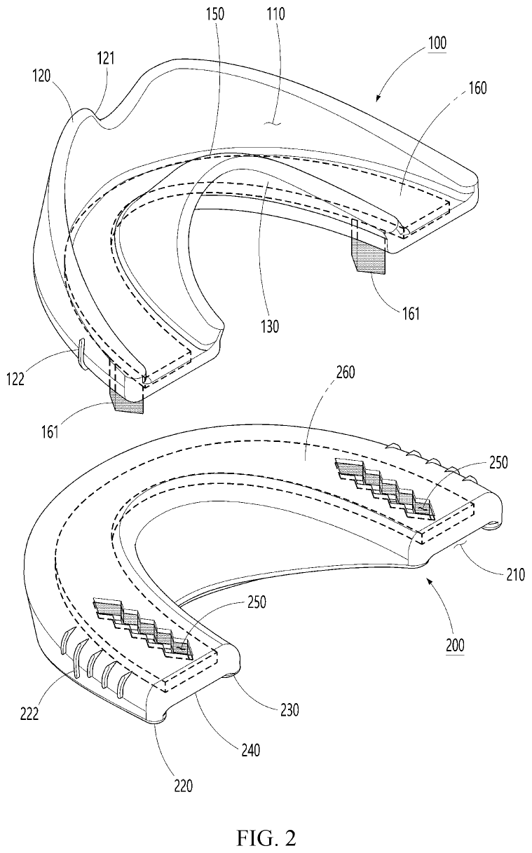

Referring to FIGS. 1 and 2, the upper mouth 100 includes the upper outer guide 120 formed along the outer edge and the upper inner guide 130 formed along the inner edge so as to form the upper teeth fitting part 110 to which the upper teeth are fit. In this instance, the upper occlusal surface 140 is formed between the upper outer guide 120 and the upper inner guide 130, so that the surface of the upper teeth occluding to the lower teeth gets in contact with the upper occlusal surface 140. Sliding rods 161 are formed at both sides of the upper occlusal surface 140. The sliding rods 161 are inserted into guide slits, which are elongated in the back-and-forth direction, so as to perform a back and forth sliding.

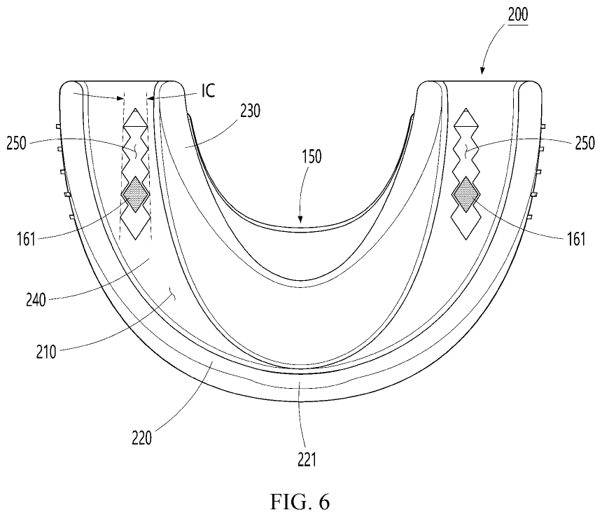

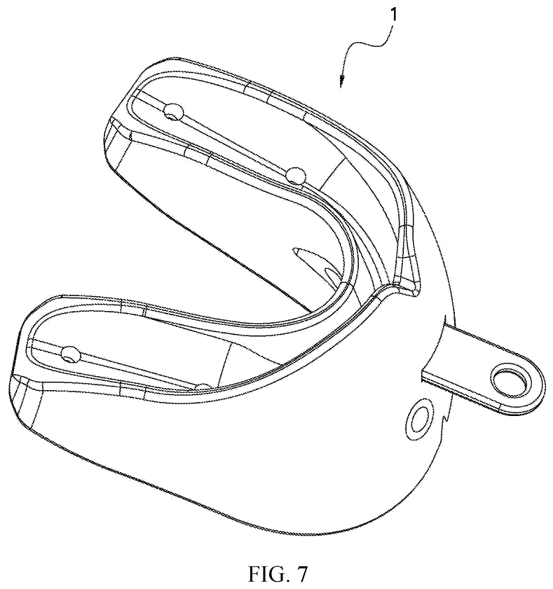

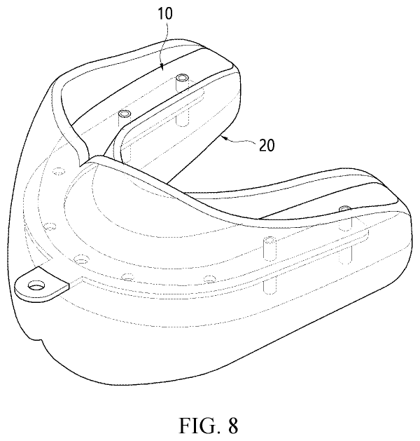

Referring to FIG. 6, the front sides of the guide slits are formed with a slope (IC) of 2° to 4° toward the center of the upper mouth 100, so that the sliding rods 161 are transformed elastically to keep the combined state with the guide slits when the upper mouth 100 or the lower mouth 200 moves back and forth. Therefore, the guide slits raise the binding force between the upper mouth 100 and the lower mouth 200. Furthermore, the guide slits are formed in zigzags in a longitudinal direction so as to be adjusted by stages. Preferably, the sliding rods 161 have a diamond shape in cross section. As described above, as an example, the sleep mouthpiece according to the present invention can not only adjust the upper mouth 100 and the lower mouth 200 back and forth but also adjust binding force since having uneven parts like the zigzag shape. Meanwhile, FIGS. 2 and 6 illustrate the shape of the guide slits and the cross-sectional shape of the sliding rods 161 just as an example. Accordingly, the sliding rods may be in a cylindrical shape and the guide slits may have the shape corresponding to that of the sliding rods, and other various combinations in shape between the sliding rods 161 and the guide slits are possible.

Referring to FIGS. 1 and 2, an upper frenum receiving groove 121 is formed at the front center of the upper outer guide 120 to protect the upper labial frenum. A single upper nonslip rib 122 which is elongated vertically is formed at each of the lower portions of the right and left sides of the upper outer guide 120 in order to prevent separation of the upper mouth 100 mounted in the mouth by getting in contact with both sides of the intraoral cheek. In this embodiment, the single upper nonslip rib 122 is mounted at each side of the upper outer guide, but may a plurality of the upper nonslip ribs 122 may be mounted.

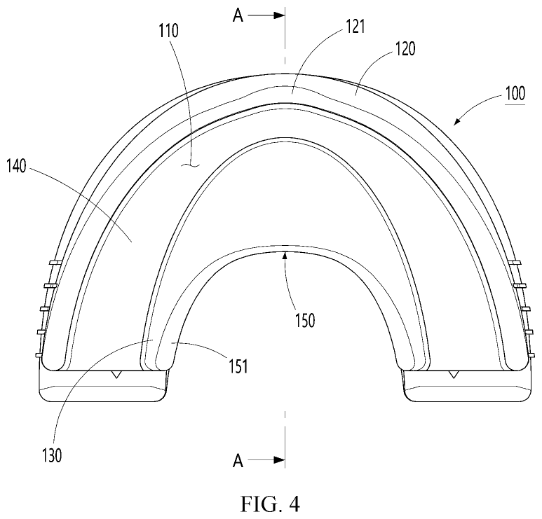

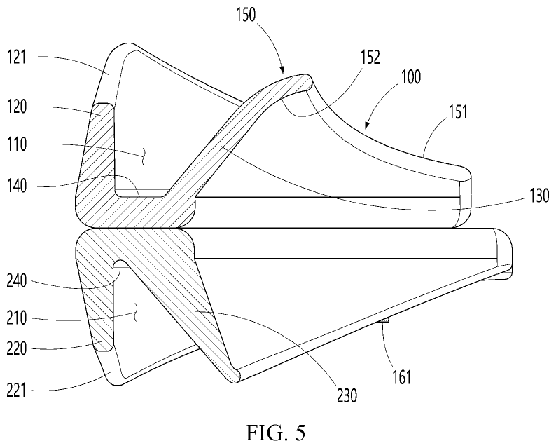

Moreover, referring to FIGS. 1 and 4, a hard palate support 150 is integrally connected to the upper end of the upper inner guide 130 to support the hard palate in the mouth to get in contact with the hard palate. Therefore, the upper mouth 100 is not separated from the upper teeth and can keep the fit condition when the end of the tongue pushes the upper inner guide 130 out of awareness during sleeping. As shown in FIG. 4, an outer end portion 151 of the hard palate support 150 is smoothly curved toward both ends from the center of the upper end of the upper inner guide 130, and an inner face 152, as shown in FIG. 5, has a curvature continued from the inner face of the upper inner guide 130 to the hard palate in order to provide the user with comfort when the user wears it onto the upper teeth.

FIG. 2 is an exploded perspective view showing an exploded state of the upper mouth and the lower mouth of the sleep mouthpiece according to the preferred embodiment of the present invention. An upper rigid panel 160 and a lower rigid panel 260 are respectively inserted into the upper mouth 100 and the lower mouth 200. Referring to FIG. 2, the upper rigid panel 160 is integrally formed in the upper inner guide 130 by insert injection in order to keep the framework of the upper mouth 100 and to keep the form transformed according to the teeth structure. The sliding rods 161 are integrally formed on the upper rigid panel 160, and are inserted into the guide slit. The upper rigid panel 160 is made of polypropylene (PP) and can maintain a gap of 5 mm to 10 mm between the upper teeth and the lower teeth.

In the meantime, the lower rigid panel 260 is substantially the same as the upper rigid panel 160, but has a communication hole 250 of the same shape vertically continued from the lower mouth 200. Furthermore, guide slots may be identically formed in the communication hole 250 or a through hole of the lower mouth 200 having the same shape as the communication hole 250.

The lower mouth 200 has the lower teeth fitting part 210 to which the teeth bridge and the lower teeth including the front teeth, the canine teeth and the back teeth of the lower gum are fit. Such a lower mouth 200 is improved in hygiene and does not make wound in the mouth since being made of ethylene vinyl acetate (EVA) like the upper mouth 100.

Referring to FIG. 6, the lower mouth 200 includes the lower outer guide 220 formed along the outer edge and the lower inner guide 230 formed along the inner edge so as to form the lower teeth fitting groove to which the lower teeth are fit. In this instance, the lower occlusal surface 240 is formed between the lower outer guide 220 and the lower inner guide 230, so that the surface of the lower teeth occluding to the upper teeth gets in contact with the lower occlusal surface 240. Sliding rods 161 are formed at both sides of the upper surface opposite to the lower occlusal surface 2140 in the vertical direction. The sliding rods 161 serve to connect the upper mouth 100 and the lower mouth 200 to be moved in the back-and-forth direction since being fit to be slidable along the guide slits of the lower mouth 200. Additionally, the sliding rods 161 serve to vertically connect the upper mouth 100 and the lower mouth 200 to be detachable since being fit to the guide slits. That is, the coupling of the guide slits and the sliding rods 161 has the function to connect the upper mouth 100 and the lower mouth 200 to be movable in the back-and-forth direction and the function to connect the upper mouth 100 and the lower mouth 200 vertically.

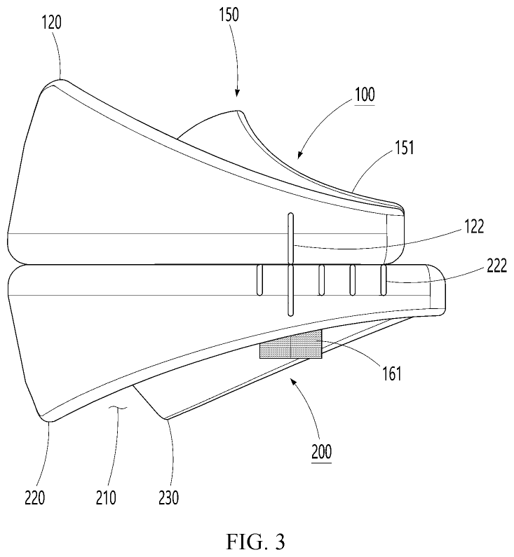

In other words, referring to FIGS. 1 to 3 and 6, a lower frenum receiving groove 221 is formed at the front center of the lower outer guide 220 to protect the lower labial frenum. A plurality of upper nonslip ribs 222 which are elongated vertically are formed at the upper portions of the right and left sides of the lower outer guide 220 in order to prevent separation of the lower mouth 200 mounted in the mouth by getting in contact with both sides of the intraoral cheek. In this embodiment, a plurality of the nonslip ribs 222 are mounted, but a single lower nonslip rib 222 may be mounted at each side of the lower outer guide.

A lower rigid panel 260 is integrally formed in the lower inner guide 230 by insert injection in order to keep the framework of the lower mouth 200 and to keep the form transformed according to the teeth structure. The lower rigid panel 260 is made of polypropylene (PP) like the upper rigid panel 160 and can maintain a gap of 5 mm to 10 mm between the upper teeth and the lower teeth.

The user forcedly fits the sliding rods 161 of the upper mouth 100 into the guide slits of the lower mouth 200, and then, adjusts the upper mouth 100 and the lower mouth 200 while moving them in the back-and-forth direction according to the user's teeth arrangement.

The sleep mouthpiece according to the present invention can be obtained through the steps of soaking the upper mouth 100 and the lower mouth 200 in hot water of 90° C. for 30 to 50 seconds and taking out of the water, and comfortably fitting and molding the upper mouth 100 and the lower mouth 200 to the user's teeth arrangement in such a way that the lower mouth 200 are pulled about 5 mm to 6 mm relative to the upper mouth 100. The steps may be repeated two times or three times till the user feels full comfort.

In this instance, the impression taking is completed when the mouthpiece is washed in cold water two or three times for ten minutes, and the user can use the sleep mouthpiece according to the present invention in order to prevent snoring and sleep apnea.

Therefore, the sleep mouthpiece according to the present invention can prevent separation of the upper mouth 100 by the power of the tongue to push out of awareness during sleeping since supporting the intraoral hard palate while getting in contact with the intraoral hard palate, and can be adjusted according to various kinds of teeth arrangements since the upper mouth 100 and the lower mouth 200 can move back and forth by coupling of the sliding rods 161 and the guide slits.

What is claimed is:

1. A sleep mouthpiece comprising: an upper mouth having an upper outer guide, an upper inner guide and an upper occlusal surface to form an upper teeth fitting part to which the upper teeth are fit, so that an upper rigid panel is inserted into the upper mouth; and a lower mouth having a lower outer guide, a lower inner guide and a lower occlusal surface to form a lower teeth fitting part to which the lower teeth are fit, so that a lower rigid panel is inserted into the lower mouth,
wherein a hard palate support is connected to the upper end of the upper inner guide and comes into contact with the intraoral hard palate to support the intraoral hard palate,
wherein sliding rods are formed at both sides of the upper occlusal surface in order to connect the upper mouth and the lower mouth with each other so that the upper mouth and the lower mouth can be moved back and forth, and
wherein the sliding rods are inserted and guided into guide slits formed at both sides of the lower occlusal surface.

2. The sleep mouthpiece according to claim 1, wherein the front sides of the guide slits are formed with a slope of 2° to 4° toward the center of the upper mouth.

3. The sleep mouthpiece according to claim 2, wherein the guide slits are formed in zigzags in a longitudinal direction.

4. The sleep mouthpiece according to claim 1, wherein an outer end portion of the hard palate support is smoothly curved toward both ends from the center of the upper end of the upper inner guide, and an inner face of the hard palate has a curvature continued from the inner face of the upper inner guide to the hard palate.

5. The sleep mouthpiece according to claim 1, wherein a plurality of upper nonslip ribs which are elongated vertically are formed at the lower portions of both sides of the exterior of the upper outer guide to get in contact with both sides of the intraoral cheek.

6. The sleep mouthpiece according to claim 1, wherein a plurality of lower nonslip ribs which are elongated vertically are formed at the upper portions of both sides of the exterior of the lower outer guide to get in contact with both sides of the intraoral cheek.
